(19) United States
(12) Patent Application Publication
(10) Pub. No.: US 2011/0173028 A1
(43) Pub. Date: Jul. 14, 2011

(54) METHOD AND APPARATUS FOR SUPPLY CHAIN MANAGEMENT

(75) Inventor: Travis Bond, Lutz, FL (US)

(73) Assignee: Eclipsys Practice Solutions, LLC, Irvine, CA (US)

(21) Appl. No.: 12/678,577

(22) PCT Filed: Sep. 17, 2008
(86) PCT No.: PCT/US08/76611

§ 371 Date: Jan. 4, 2011

Related U.S. Application Data
(60) Provisional application No. 60/972,917, filed on Sep. 17, 2007.

(51) Int. Cl. G06Q 10/00; G06Q 30/00

(52) U.S. Cl. 705/3; 705/28; 705/26.8; 705/22; 705/400; 705/303

(57) ABSTRACT

A method and system for managing inventory for an ancillary medical facility that can be integrated into existing electronic healthcare records systems or practice management systems. The invention enables clinician users to order medical supplies electronically and to track the entire supply chain of the inventory item from the vendor/manufacturer to the patient. In one embodiment, requisitions are automatically generated when an inventory supply falls to a critical volume or reorder point. The system of the invention provides reporting modules that allow clinician users to analyze cost, inventory usage and adjustment, and supply conflict.

This application is based on, and claims priority to, U.S. provisional application No. 60/972,917, having a filing date of Sep. 17, 2007, and entitled Method and Apparatus for Supply Chain Management.

### BACKGROUND OF THE INVENTION

The delivery of medical supplies, durable medical equipment (DME), medical devices and pharmaceuticals in this country is an inefficient system today. Delivery is essentially a manual process. It requires human intervention at multiple stages. Physician practices are visited by sales staff from the distributor or a phone call is placed by the distributor to obtain the order from the physician practice. A paper purchase order is generated. This purchase order is hand-keyed into the distributor's system to execute the transaction. The order is transmitted to the distributor's warehouse to determine if the distributor has the necessary stock to fulfill the customer's order. If the distributor is out of stock of any item from the customer's order, the distributor must generate a purchase order which is transmitted via facsimile to the manufacturer to execute the order. The manufacturer transmits the order to its warehouse to fulfill the order and ships the product to the distributor or directly to the customer depending on contractual arrangements. A bill is then generated by the manufacturer and mailed to the distributor. A second bill is generated by the distributor and mailed to the customer. There are many opportunities for errors and miscommunications in this process. Conventional systems employ a series of wholesale and retail corporations that retail medical supplies, durable medical equipment, medical devices and pharmaceuticals to healthcare providers, hospitals, ambulatory surgical centers (ASCs), pharmacies, IPA (Independent Physician Association organizations and RHIOs (Regional Health Information Organizations). Elaborate networks of warehouses, sales staff, inventory and ordering systems (many of which are still paper based) are required to market to the customer, place the order with the manufacturer (sometimes indirectly) and get the product back to the customer. None of these systems is interoperable or able to communicate with one another so the manufacturer has no way to forecast long-term what products will be required. Conventional systems are not integrated with practice management (PM) and electronic health records (EHR) systems. Ordering on these systems provides links only to distributors' or manufacturers' websites, not to American National Standards Institute (ANSI) based communications. Furthermore, nothing in the current technology provides for tracking the actual delivery and use of medical items at the patient level.

### SUMMARY OF THE INVENTION

It is therefore an objective of the present invention to provide a novel method and apparatus to automate the ordering, processing and delivery of medical supplies, durable medical equipment, medical devices and pharmaceuticals. It is another objective of the invention to provide an inventory system that may be integrated into existing practice management (PM) and/or electronic health record systems. It is another objective of the invention to provide a system that allows for tight control of the medical product and pharmaceuticals supply chain from the manufacturer's level to the patient's level.

The invention contemplates a method of and system for supply chain management for an ambulatory medical facility. The invention includes a method of managing a supply chain that tracks the goods from manufacturer to end user and a web-based application to carry out the methods described herein. The invention also includes an inventory module that can be integrated to a patient management or electronic health record systems.

According to the present invention, a novel method of managing medical supplies, durable medical equipment, medical devices and pharmaceuticals and a web-based system for implementing such method is used for automating and integrating the ordering, processing and delivery of medical supplies and pharmaceuticals from the manufacturer level to the patient level. The system is preferably an ASP.net web-application and uses security features that are compatible with the ASP.net architecture such as login pages, user authentication and user authorization and role assignment to restrict web usage. Communications between the manufacturers, vendors, and clinicians will be through secure internet connections, preferably supported by an EDI server. Information about the medical supplies or pharmaceutical items is preferably stored on a database server. Another objective of the invention provides a system that allows the user to create case costing based on information provided by the practitioner as well as consideration for the fixed cost and direct labor.

The process of the invention enables manufacturers and vendors to communicate directly with the healthcare provider regardless of the clinical setting. By removing the middle men, this process removes in excess of $20 billion dollars in costs from the supply chain. Embodiments of the invention also make possible the ancillary benefit of allowing retail pharmacy chains, healthcare product manufacturers and others to get out of the distribution business, a goal these establishments have wanted to accomplish for many years. This process, which focuses on the practitioner-to-vendor direct communication would allow for example, Cardinal Health, McKesson, and others to close their own distribution centers (DCs) and rely on a third party for the delivery of merchandise directly from the manufacturer to their own outlets.

The method of the invention removes the preparation of purchase orders from a manual process to an electronic process. In so doing, order processing time is reduced by 50-90% per purchase order. The reduction in time provides more time for the manufacturer and practitioner to focus on value-added initiative. The process also eliminates charge-backs due to direct interact to P+Q for contract and/or negotiated pricing with healthcare entities. It enhances data accuracy, which directly relates to significant decreases in Days Sales Outstanding. When purchase orders and invoices are accurate, invoice reconciliation time is reduced and a decrease in Days Sales Outstanding is realized. Improving accuracy of orders reduces customer service efforts to handle purchase orders; inventory management and stock on hand requirements can be easily managed and invoice discrepancies replenished with minimal stocking levels that are automated. More accurate orders lead to a decrease in number of credit/re-bills and returned goods. Greater accuracy in electronic orders positively impacts Days Sales Outstanding, as accurate invoices are paid prior to ones with discrepancies.

By partnering with a major financial partner who will provide a Merchant Account such as Visa, MasterCard or American Express and/or procurement cards, healthcare providers can achieve a greater than seven day float (typically 35+). This allows greater flexibility for the financing of inventory for the provider since insurance payers historically take 30 or more days to pay healthcare claims. Another advantage of the process is that the direct interface between the vendor and practitioner can eliminate cold calls from vendors who are suppliers or distributors to the practitioner, thus increasing the amount of time the practitioner can give their patients thereby increasing their billable hours.

Further embodiments of process include the additional steps of downstream tracking. Downstream tracking allows for tracking to the level of the actual patient who received the medical item. The capability of tracking medical supplies to the patient level enhances patient safety by utilization of such items in a patient population and aiding in recalls.

The invention further provides an inventory management system that is configured to integrate with a PM or EMR. The system enables a user to create electronic requisition and purchase orders. In one embodiment, the system automatically generates a requisition when an inventory item falls below a critical volume or reorder point.

### BRIEF DESCRIPTION OF THE DRAWINGS

The details of one or more embodiments of the invention are set forth in the accompanying drawings and the description below. Other features, objects, and advantages of the invention will be apparent from the description, the drawings, and the claims.

FIG. 1 shows an inventory work flow diagram according to an illustrative embodiment of the invention.

FIG. 2 shows an ordering workflow diagram according to an illustrative embodiment of the invention.

FIG. 3 shows a catalog updating workflow diagram according to an illustrative embodiment of the invention.

FIG. 4 shows a screenshot of the Admin screen of the inventory management module according to an illustrative embodiment of the invention.

FIG. 5 shows a screenshot of the Inventory Preferences Configuration screen of the inventory module according to an illustrative embodiment of the invention.

FIG. 6 shows a screenshot of the Inventory Categorization Details screen of the inventory module according to an illustrative embodiment of the invention.

FIG. 7 shows a screenshot of the Add/Edit Inventory Items screen of the inventory module according to an illustrative embodiment of the invention.

FIG. 8 shows a screenshot of the Physical Count Worksheet screen of the inventory module according to an illustrative embodiment of the invention.

FIG. 9 shows a screenshot of the Purchase Order Details screen of the inventory module according to an illustrative embodiment of the invention.

FIG. 10 shows a screenshot of the Alert Configuration screen of the inventory module according to an illustrative embodiment of the invention.

FIG. 11 shows a screenshot of the Purchase Order Receiving Details screen of the inventory module according to an illustrative embodiment of the invention.

FIG. 12 shows a screenshot of the Physician Preference Card Details screen of the inventory module according to an illustrative embodiment of the invention.

FIG. 13 shows a screenshot of the CENTCOM screen of the inventory module according to an illustrative embodiment of the invention.

FIG. 14 shows a screenshot of the Appointment Details Calendar Tool Tip according to an illustrative embodiment of the invention.

FIG. 15 shows a screenshot of the Preference Card screen accessed from the Pref Card Icon in the Calendar Tool Tip according to an illustrative embodiment of the invention.

FIG. 16 shows a screenshot of the Inventory Summary View Box in a patent chart according to an illustrative embodiment of the invention.

FIG. 17 shows a screenshot of the Patient Inventory Allocations Encounter screen according to an illustrative embodiment of the invention.

FIG. 18 shows a screenshot of the Patient Inventory Allocations screen according to an illustrative embodiment of the invention.

FIG. 19 shows a screenshot of the Intellicharge screen according to an illustrative embodiment of the invention.

FIG. 20 shows a screenshot of the Costing Details screen according to an illustrative embodiment of the invention.

### DETAILED DESCRIPTION OF THE INVENTION

In an embodiment of the process of the invention a user with appropriate rights creates the requisition for the desired inventory items and submits the requisition. An electronic alert is automatically triggered to notify a user authorized to process the requisition and create a purchase order. The user creates the purchase order, which preferably is transmitted electronically to the vendor via an electronic data interchange (EDI) server. The vendor receives the purchase order and sends confirmation electronically, again through the EDI server, back to the user. The vendor processes the order and prepares the medical inventory items for shipment. When the order is ready for delivery, the vendor uses a controlled logistic delivery service such as United Parcel Services (UPS), Federal Express or the United States Postal Service. The ability for the system to post electronic orders directly to the manufacturer and the use of a controlled logistic delivery service enable direct tracking of the medical item by the provider. The vendor notifies the user of the shipment and provides the shipment tracking number. At the same time, the vendor transmits an invoice directly back to the user's practice's accounting system via a direct interface to an accounting system, such as QuickBooks. In another embodiment, the process includes the step of interfacing directly with the user's medical billing systems.

The user records the tracking number in the practice's inventory module. When the shipment arrives, the user checks-in the inventory items using the practice's inventory module. If the order includes items that have serial numbers, lot numbers or other identification numbers, the user enters that identification into the inventory module. If the user has received all the items on order, the user marks the order as complete. If not all ordered items were received, the user notifies the vendor electronically and changes the order's status to either partially complete or incomplete.

If the order is complete, the user makes on-line payment through on-line merchant account vendors, such as large financial institutions including Visa, MasterCard or American Express. In another embodiment, payment is made through a corporate procurement card or purchasing card programs.

The process of the invention further comprises the steps of making patient encounter appointments and preparing for them. In response to a scheduled patient encounters, the user relies on Physician Preference Cards to prepare the clinical setting. Physician Preference Cards provide information on what inventory items are needed for a particular physician, patient chief complaint, and reason code. The user selects the items that are required for that patient encounter and adds those inventory items to the patient record. The user then pulls those inventory items to ready for the patient encounter.

The process of the invention further comprises the steps of closing the patient encounter using the inventory module at the point of sale, and billing the patient. At the conclusion of the patient encounter, the user locates the record of the encounter, makes adjustments for the inventory items used and charges for the supplies used. In other words, the user checks off the inventory items that were used during the encounter and saves that information as an inventory allocation record. The unused items are returned to inventory automatically. The user also adds the price of the inventory items and fixed costs to the charge. The user then selectively either bills the patient for the encounter or bills the insurance carrier for the encounter. In another embodiment of the process, the further step of making an appointment for the next patient encounter is included.

In another embodiment of the invention, the process includes the initial steps of setting up the preferences for the account. In an embodiment where the practice comprises multiple locations, an authorized user will set up the various departments and storage locations. The user then adds their vendors' information to the system, and proceeds to manually add the vendors' inventory items that are used by the practice to the system. In another embodiment, the user queries the vendors' electronic catalogs for inventory items information. Preferably, the vendors' catalogs reside on an EDI server and are maintained by the vendors.

In another embodiment of the invention, the requisition is not manually created. Rather, as the inventory count of that item falls to a pre-determined critical volume or reorder point, a requisition is automatically generated and submitted.

Alternately, where a practice has a partnership with a manufacturer, the order transmission will be directly with the manufacturer of the inventory item, thereby bypassing the distributor and resulting in even greater savings and efficiency.

In another embodiment, the process includes the step of specifically searching electronic catalog databases to select, order and bundle these supplies and to manage inventories using a web-based application (ASP hosted) that relays data between purchaser, manufacturer and a financial institution. The inventive process includes routing requests for purchase and shipment, and billing (invoices and statements) directly from the manufacturer or vendor. A further embodiment of the process includes steps to identify price discrepancies at the point of acknowledgement. Identifying discrepancies prior to the point of invoice also dramatically reduces Days Sales Outstanding.

In a further step of the process, the user creates the Physician Preference Cards for the practice. The preference cards may be set up for a location, a physician or a reason code. The user enters information about the physician, the procedure or the diagnostic test and the necessary or preferred equipment. The preference cards are used to promote the efficiency in setting up for patient consultations encounters, examination rooms, operating rooms, etc. Nurse practitioner users could use the preference cards to pull inventory items to prepare for a patient appointment or an operation. Thus the process of the invention comprises the further step of using preference cards to prepare for patient encounters.

In a further embodiment of the invention, the process further includes a record-keeping step for the tight control of the supply chain. The process tracks all transactions from the inception of the practitioner's order to the shipping and delivery and finally to the dispensing of or application to the patient. Such tracking advantageously gathers all information to comply with the requirements of the state Pedigree Acts and the pedigree requirements of the Prescription Drug Marketing Act on drug distribution.

The invention also includes a process for responding to recalls of medical products, equipment or pharmaceuticals. Once the user receives notice that a recall has been issued for the inventory product, the user can search its inventory module to see if the practice had used the recalled item. If the user establishes that the practice used the recalled item, then the user will search through the inventory allocation records to locate the patients who have been exposed to the recalled item. The process includes the step of notifying the patients of the product recall.

The invention includes a system which is capable of carrying out the method disclosed above; i.e. to enable electronic ordering of medical supplies; monitoring of inventory and requests, purchase orders and overall supply chain management. The system of the invention is preferably compatible with ASP.net architecture. The system of the invention is preferably installed on the clinician user's computer system that already has an electronic healthcare records (EHR) or practice management (PM) system installed. In another embodiment, the system of the invention is installed on the practitioner user's computer system as a stand-alone program not interfaced with an EHR or PM system. The user's computer system is preferably operationally linked to an electronic data interchange (EDI) server capable of transmitting data between the practitioner's and the vendor's computer system. The EDI server is preferably configured to handle batch processing of data and is further configured to carry out secured communications between partnering practitioners, manufacturers and vendors that at least meet ANSI and HIPAA standards.

An illustrative embodiment of the invention preferably has a unique trade dress, configurability administration and ASP.net architecture for inventory ordering and management of the supply chain for medical supplies. The ability to bridge applications that support the medical community and centrally managed inventory is unique.

The system refines core business processes to improve customer service on the manufacturer side and decrease transaction costs by accelerating transition from manual to electronic processes and increasing product and order status information available to customers and trading partners. It allows for improved accountability for rebating purposes and eliminates or minimizes the rebate “gaming” that manufacturers currently experience. The system's intelligence minimizes the need for manual intervention on electronic orders.

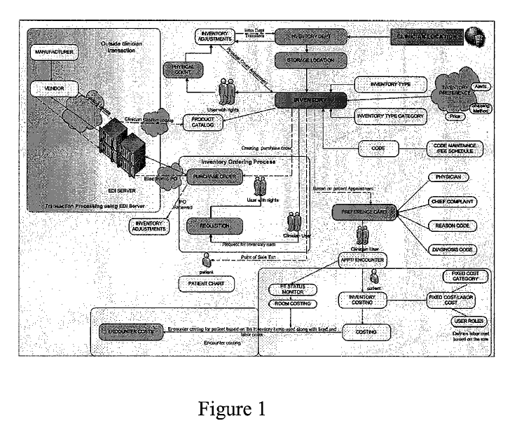

FIG. 1 depicts inventory workflow, which involves processes on the manufacturer and vendor level and processes on the clinician user level. In an illustrative embodiment of the invention, manufacturers transmit information about their products such as unit quantity, pricing and shipping requirements to their vendors. The vendors create an electronic catalog or upload the information to an existing catalog to ensure that the information is current. In a preferred embodiment, the vendors' electronic catalogs reside on the EDI server or a parallel database server. Alternately, the manufacturers may create and keep up-to-date electronic catalogs that preferably reside on the EDI server or a parallel database server. In another embodiment, the vendors catalog updates are automatically communicated to the user's system, which updates the information under the “Vendor Product Catalog” tab either automatically or through manual commands.

In one embodiment, the invention system enables the clinician clients to manage their inventory. The product information of the inventory is derived or downloaded from the vendors' product catalogs. Inventory information is categorized as Inventory Type, Inventory Type Category, or Inventory Code derived from code maintenance or fee scheduled. The information is stored in a database that is accessible by the clinician user. Such information may be readily adjusted by users with rights. For example, users with rights may undergo a periodic physical count of an item and perform inventory adjustments against the database information. Such adjustments may also be carried out when intra-departmental transfers take place.

In an embodiment of the invention, inventory information is received from the inventory department and storage locations. In another embodiment, authorized users conduct a physical count of inventory items and compare the results with the inventory information on the system. Discrepancies between the physical count and the electronic information in the database are reconciled through inventory adjustments. Other inventory adjustments may be made in the event of intra-departmental transfers.

In another embodiment of the invention, authorized users may customize inventory preferences, setting alerts, preferred shipping methods for the inventory item, and pricing information. Further, alerts are triggered when the inventory count of an item falls below a predetermined count.

In another embodiment, the inventory ordering process is initiated by an alert that is triggered when the count of an item falls below a pre-determined amount. The alert results in an automatic generation of a purchase order. In another embodiment, the alert may prompt a user with rights to prepare a requisition for the inventory item(s) or to generate an electronic purchase order for the item(s). In either embodiment the electronic purchase order is transmitted via the EDI server to the vendor. The vendor acknowledges receipt of the purchase order. The vendor may process the purchase order and ship out the inventory items. In some cases the vendor may not have the items in stock. In another embodiment of the invention, the vendor communicates its order to the manufacturer through the EDI server. The manufacturer will thereafter process and ship the items to the vendor. In the preferred embodiment, the vendor will ship the items through a controlled logistic delivery service such as United Parcel Services (UPS), Federal Express or the United States Postal Service. When the items arrive at the clinician user's location, they are checked into the system and an inventory adjustment will be triggered to bring the inventory information up to date.

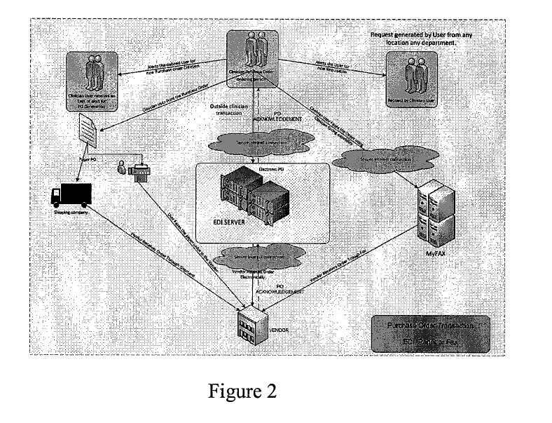

FIG. 2 depicts ordering workflow and the automated process of the invention. It also shows alternate embodiments of transmitting a paper purchase order by fax transaction. In one embodiment of the invention, the ordering process is a seamless transaction from starting point—a request generated by a clinician user—to the end point that requires only one entry of the order. A request is generated by a user from any location and any department of the practice in the form of an electronic requisition. Users authorized to prepare purchase orders are alerted to the incoming, new requisition(s) and convert the requisition(s) into purchase order(s). The purchase orders are then preferably transmitted over a secured internet connection and EDI server to the vendor(s). Upon receipt of the purchase order(s), acknowledgement is then sent back to the user, again preferably transmitted over a secured internet connection and EDI server.

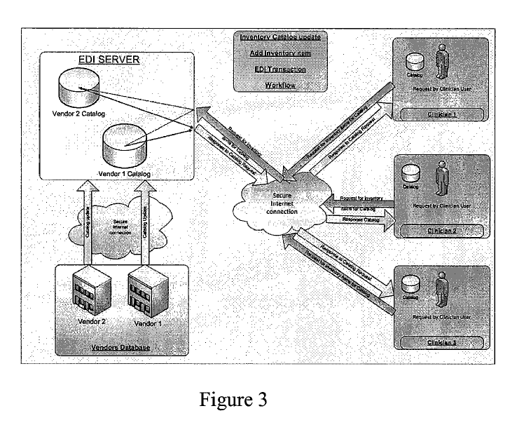

FIG. 3 illustrates the work flow of catalog updating and the EDI server transaction when users request catalog items. Vendors set up their product database or catalog on the EDI server and periodically update the information therein. Users may custom build an electronic version of the catalog for their system. Users request certain inventory items to be added to their electronic version of the catalog via a secured interne connection communication to the vendors' catalogs on the EDI server. In response, such items are added and information about the inventory items added to the electronic versions of the vendors' catalogs on the users' systems.

In the following discussion of the system of the invention, it is understood that when a change is made to the various components of the system, the user will save the changes by clicking the save button. Likewise, it should be understood that the user can terminate a transaction by clicking the cancel button.

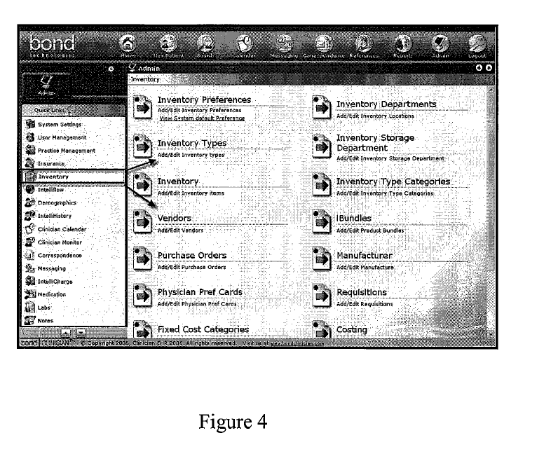

Referring to the drawings, FIG. 4 shows the ADMIN screen of an illustrative system of the invention. The graphical user interface allows the end user to easily navigate through all of the set up and configuration steps.

### Setting Up Preferences and Defaults

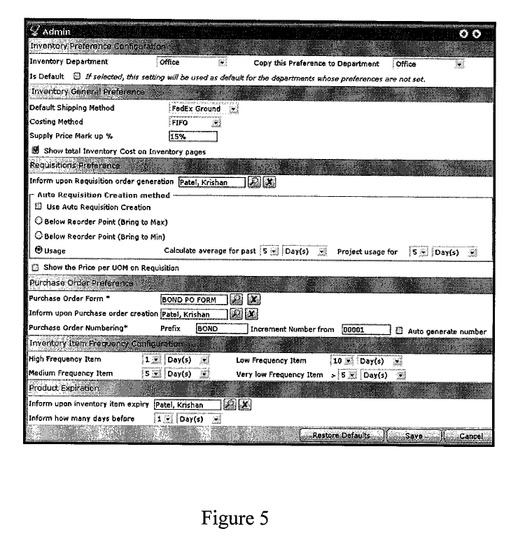

During the initial set up, the user logs into the inventory module interface using a user id and password at a computer terminal. The user navigates to the ADMIN panel of the module (FIG. 4) and clicks on the Inventory Preferences link to be directed to the Inventory Preference Configuration screen (FIG. 5). In that screen, the user sets up either the preferences or any preferred default settings for the practice department, or both.

The user may choose a default shipping method, costing method and supply price mark-up percentage for its inventory. The drop-down menu for shipping method includes at least the following default shipping methods: by UPS, USPS, UPS Ground, FEDEX, FEDEX Ground, and DHL. In other embodiments, the user can add new shipping methods to the drop-down menu. Likewise, the drop-down menu for costing methods includes at least the following: FIFO (first in first out), where the first inventory to come in goes out first, and cost is defined thereby; LIFO (last in first out), where the last inventory to come in goes out first, and cost is defined thereby; and AVG Cost, which calculates the cost based on average of all the available inventory cost.

For requisitions preferences, the user can choose to use the automatic requisition generation feature and determine the critical volume, or reorder point, for the inventory item. The user can also set the automatic requisition to bring the inventory to the maximum count, to Par, or to average usage based on the average past and projected future usage. For purchase orders, the user can choose the format of the order and customize the P.O. numbers. More importantly, the user can choose the individual who will receive notice that a purchase order has been generated in the “Inform Upon Purchase Order Generation” box. The user may also elect whether to have the price shown with the unit or measurement or not. The user can define the frequency of inventory items and appoint one to receive notice that the product will soon expire.

For the embodiments where the user has set up different departments for the practice, preferences can be applied to other departments by clicking “Copy this preference” in the department dropdown menu. Users may restore the default settings by clicking the “Restore Default Preferences” button. In another embodiment, the user may set up user permissions and global (system-wide) preferences. Reorder points are set in this area—a purchase order is automatically generated when the quantity of an item falls below the designated quantity.

Once the set up is complete, the user saves the selections or changes and closes the screen. The user may then navigate to a different menu or log out.

### Creating Departments and Storage Locations

From the ADMIN screen, the user may navigate to the Inventory Department screen. The Inventory Department screen displays a list of inventory departments and clinician locations with their address and account code. The user may remove an inventory department by deleting the entry.

The user may add a new inventory department by clicking on the “NEW” button, which brings up the “Inventory Department Details” screen. The user enters the name of the new department and chooses an associated location and facility. For example, the user may configure different departments for different physical branch locations, by practice specialty, or by physician practice. Textboxes and drop menus are available for the user to enter information that is unique to the department such as account code, address, a contact person and telephone number. The user may also enter the preferred shipping address and invoice for the department. The user then saves the new inventory department entry.

Similarly, the user can set up a new the storage location once a department has been created. From the ADMIN screen, the user clicks on the “Inventory Storage Location” button, which opens up a screen by the same name. The screen displays a list of storage locations and their associated inventory department. The user may remove any storage location by deleting it from the list. By clicking “New,” the user is able to pull up the “Inventory Storage Location Details” screen to enter a new storage location and inventory department.

### Defining Inventory Types

As part of the set up process, the user is required to enter inventory items. The first step is to set up the inventory types. From the Admin section, the user navigates to the Inventory Type section. The screen displays all the existing inventory types by name, and whether they are system or user defined. There are four system-defined inventory types: Supplies, Instruments, Equipments and Medications. The user may customize the inventory types to the practices' needs by creating additional user-defined inventory types. Clicking on the “New” button will transfer the user to a pop up screen to enter the names of the user-defined inventory type and associate it with a system-defined inventory type. For example, the user can add “Food Items” as an inventory type. If any inventory type is removed by clicking the “Delete” icon on the right hand column, it will be soft deleted and will be removed from the choices in the drop-down menus from the system. If the user attempts to delete any inventory type that continues to have inventory items associated with it, an error message will be displayed to warn the user that dependencies to the inventory type exists and that it cannot be deleted until those dependencies are removed. The “Inventory Add” screen allows the user to associate any inventory item with an inventory type and fill out other relevant information about the item. The user can save the item under the inventory list.

In another embodiment, the user adds the inventory item from the vendors' electronic catalog. From the Admin screen, the user clicks on the Inventory link and navigates to the Vendor Product Catalog page. By selecting the inventory items there the user can add them to the inventory; however, the user must still associate an inventory type and inventory category with the item.

### Defining Inventory Categories

To further classify inventory items, the user may create multiple inventory categories by clicking on the “Inventory Type Categories” icon from the ADMIN screen. The user is directed to the “Inventory Type—Categories” screen, which displays a list of existing inventory categories. An inventory category filter allows the user to search categories by name and inventory types. Search results are displayed in the bottom frame of the screen. The user can customize the number or records displayed per page.

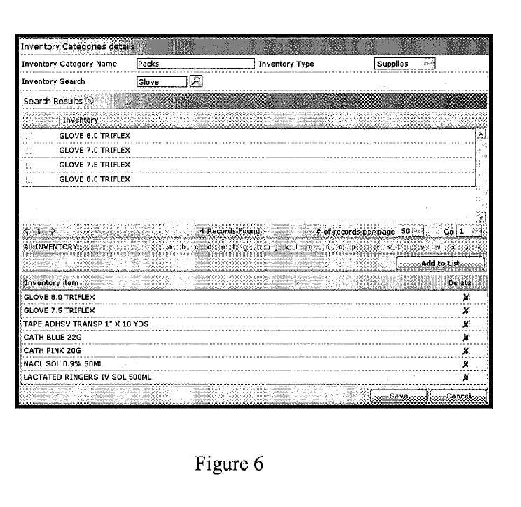

By clicking on the “New” button, the Inventory Categories Details screen (FIG. 6) appears that allows the user to create a new or edit an existing inventory category. The category must be associated with an inventory type: when creating a new category, the user must select an inventory type from a drop-down menu. If a later need arises to change the inventory type associated with a category, the user must ensure that no inventory items are associated with the category. Only then will the user be permitted to dissociate an inventory type from a category after it has been set up.

Once the inventory category has been set up, the user can add or associate the category with inventory items. FIG. 6 illustrates this process. The user can search for inventory to associate with the category by searching for either inventory type or inventory item name, or both. The user will enter the category name and select those inventory items from the search result list that are to be added to the category. By clicking on the “Add to List” button, the user adds the selected items to the Inventory List in the bottom grid. The user can disassociate an inventory item with the category by clicking on the delete button in the right hand column of the bottom grid.

In another embodiment of the invention, a further search box is provided to enable the user to filter out inventory items associated with at least one category.

### Adding Inventory

In the Inventory section (ADMIN>>Inventory), an inventory filter is provided for het user to quickly locate the item of interest. The user can search by inventory name, category, inventory type and product code. For example, the user may enter a user-defined category of “OR Misc” and the inventory type of “Supplies” to pull up inventory items. The resulting list displays the inventory item name, category, inventory type, quantity on hand, the manufacturer or vendor part number, and re-order point. The user can also view the item's history to see when the item was ordered in the past and how much the practice paid for the item. Inventory items can be removed from the list by deleting the entry.

When the user first navigates to the main inventory screen, the bottom part of the screen remains blank until the user locates an item of interest through the Inventory Filter. The user can continue to add to the inventory list by entering different criteria in the Filter. If the stock of the inventory item has fallen below a critical volume or reorder point, the entry will appear in red to visually set it apart from the other entries so that it is easily identifiable. The inventory list will include the item name, the item category, the inventory type, the last updated date and the number of stock on hand. Users with rights can optionally view the total cost of the inventory.

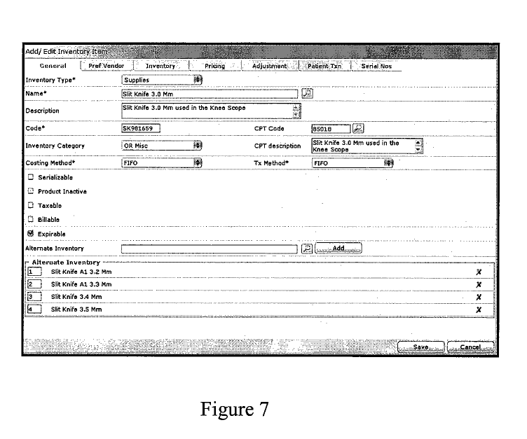

By double-clicking on the inventory item, the user is directed to the Add/Edit Inventory Items screen (FIG. 7). The user is likewise directed to this screen when he or she clicks on the “New” icon in the Inventory screen. The screen contains seven (7) separate tabs: General, Pref Vendor, Inventory, Pricing, Adjustment, Patient Txn, and Serial Nos. Under the General tab, the user adds the basic item information: inventory type, item name, item description, inventory category, item code, CPT code, CPT description, costing method, transaction (Tx) method.

The user may also identify whether the product is serializable, inactive, taxable, billable or expirable. If the item can be serialized, the user will be prompted to enter the serial number and/or lot number whenever the practice receives this item as part of the receiving procedure. If the item is expirable, the user will be prompted to enter the expiration date. As the date nears, the user will be alerted to the upcoming expiration. If an item is identified as inactive, the product will not available in preference cards or any search for the inventory. If an item is identified as taxable, that will be reflected in any purchase order or point of sale. Likewise, a “billable” item is tracked as such for insurance billing and point of sale charge purposes.

The user can also define any alternate inventory for that specific item that may be acceptable functional substitutes. The user would enter ranking information to see how closely the alternate inventory conforms to the actual item.

The user saves the foregoing information in the Inventory General tab screen. By clicking on the “Pref Vendor” tab, the user navigates to the Vendor Details screen to view the vendor list available for that inventory item. The user can designate a vendor as a preferred one by checking the “Pref” radio button. The lead time box defines how much lead time is required to fulfill the order. The Cost/Unit field is automatically populated and defines the cost of the inventory item provided by that vendor based on the last purchase order received from the vendor. The vendor item # box defines the catalog item number from vendor catalog. The Order/Unit of Measurement (Order/UOM) drop-down menu shows the unit type as defined under the “System Setting” screen in the ADMIN screen. The Quantity/Unit of Measurement (Qty/UOM) box defines what is the quantity per unit of measurement. The Contract Rate and Contract Exp Date field depend on what the defined contract rate is with the vendor and when that contract expires. The user may click the “Add” icon to open a vendor search screen to locate additional vendors that offer this item. Clicking “Remove” will delete the selected vendor from the Vendor Detail field box. The user may not remove a preferred vendor from the list without choosing another vendor as a preferred vendor. Prompts are provided to ensure that the user saves the information prior to navigating away from the screen.

The user accesses the “Inventory” tab for item specific information. The screen provides a dashboard view for inventory statistics. The user specifies the inventory item location and storage department. These fields are automatically populated from the default inventory location and storage location (the current user's defaults); however, the user can change the inventory location field to ALL. With that, the user can also choose any and all of the storage department in drop-down menu. The user can access information about the inventory item such as the number in stock, the number of items on purchase order, and the average costs. The user can set the maximum and minimum level and the reorder point for the inventory. The bottom frame shows historical information such as the last updated date, last updated quantity, who conducted the last update, last updated average cost, maximum cost and minimum cost. In the Inventory tab the user can add or edit inventory item related information, set the reorder point, and view the inventory stats based on inventory location and storage location. The user can view the current in stock values, on-purchase orders, average cost, maximum and minimum levels. The user can also view at a glance the historic information of when the last update was, in what quantity, and who updated the entries. The user can also view the last average cost, maximum and minimum costs.

The “Pricing” tab gives the user the ability to define the pricing for the inventory item by different pricing strategies. The user can choose from four different pricing models from the drop-down menu, as follows:
1. Mark up percentage from cost—The user selects what percentage of the cost he or she wants to set as the price. For example, if the cost of the item is $5 and the mark up percentage from cost 120%, then the price of the item will be (5*120/100)=$6.
2. Fixed Rate—The user can select what the price will be for the item.
3. Differential mark up percentage of cost—The user can set up a range of costs and set the percentage mark up price for that range.
4. Gross Margin—The user can enter points from which the price will be calculated based on the formula PRICE=COST/((100-POINTS)/100).

The “Adjustments” tab gives the user the ability to view the inventory adjustments done for specific inventory items. The Adjustments filter allows the user to customize his or her view. The user can enter different options in the Adjustment filter to narrow down his or her search. The user may search by entering the following information into the textboxes: adjustment type, inventory location transferred to, inventory location transferred from, storage location transferred to, storage location transferred from, and vendor. For example, the user may view any of the adjustment history for intra department transfers, physical inventory count adjustments, or Point of Sale (POS) adjustments. The user can also make adjustments in this screen for intra department transfers and physical inventory count adjustments. The user can also set automatic adjustments for Purchase Order Receiving. When the adjustment is made as a result of Purchase Order Receiving, the P.O. number is shown in the P.O. column and the Adjustment “P.O. Adjustment (+)” is shown.

When the user selects an entry in the Adjustment list and clicks on the “Adjustment” button in the bottom of the screen, the “Inventory Adjustments Details” window pops up. The entries for inventory name, inventory type, and inventory category are automatically populated. The window shows the pertinent details of the adjustments made to that inventory item. The inventory location and storage department options both default to the user's pre-defined defaults. The Inventory Details field box shows the following:
reorder point quantity for the inventory item;
the maximum level quantity for the inventory item;
the minimum level quantity for the inventory item;
the on-hand quantity for the inventory item; and
the average cost for the inventory item.
   The Adjustment Details field box allows the user to view and make adjustments to the records. A drop-down menu is present for the user to enter the “Adjustment Code.” Different code input results in some different fields. For example, if the user selects the “Intra Dept Transfer(+)” then the drop-down menus for inventory department and storage department can be changed to indicate the transfer of inventory. In another example, if the user selects the “P.O. Received (+)” code, then the P.O. textbox is activated to enter the P.O. number; and the vendor drop-down menu can be used to determine from which vendor the user received the P.O. The user can enter the Purchase Order number in the P.O. textbox. The Vendor drop-down menu shows all vendors in the system. The Inventory Location drop-down menu shows the inventory location to which the user wants to transfer the inventory item. The storage department drop-down is based on user selection of the inventory location to which the user wants to transfer the inventory item. The user enters the number of items he or she wants to transfer in the Quantity box. In addition, the user enters the unit price for the item and the adjustment date. There is also a Comment section so that the user can enter explanatory comments about the inventory adjustment.

The “Patient Transaction” tab of the inventory screen provides the user with the ability to view all patient transactions by specific patient, inventory items or inventory type. A patient transaction filter is provided with textboxes so that the user can search for a patient name, a reason code, an inventory type, or a lot or serial number. The search results are displayed in the bottom screen by transaction (tx) date, patient, quantity, reason code, and the lot or serial number. The user can also view the dispensed quantity and the transaction date.

In another embodiment of the invention, the Patient Tx Filter further comprises the ability to segregate POS transactions completed from the CENTCOM section of the invention from an encounter transaction completed in the Patient Management section of the invention.

The user can view the serial and lot numbers information in storage for an item in the “Serial Nos.” tab. A Serial No. Tab Filter is provided so the user can sort the inventory records by lot number, serial number, PO number, expiration date and the person who received the order. For example, if the user inputs a lot number in the filter, the search result will return all items with serial numbers associated with that lot number.

### Inventory (Equipment/Instrument) Edit Screen

Where the inventory item is a piece of equipment or an instrument, a further module—the Inventory (Equipment/Instrument) Edit screen—is provided for the user to manage and cost the item. The screen has four (4) tabs: general, equipment or instrument (“Equip/Instru”), pricing and patient transaction (“Pt. Txn”). In the General tab, the user can add or edit equipment or instrument, define its inventory type, and to differentiate items relevance in different tabs. Textboxes are provided for the user to add the inventory name, description and codes (including CPT codes). The user may select an inventory category for the equipment from a drop-down menu. The user can also define whether the item is active, taxable, billable, and/or expirable. If the item is expirable, the user is prompted to enter an expiration date. Further, the user may define and rank suitable alternate inventory items in case the particular piece of equipment becomes inactive or is unavailable.

In another embodiment of the invention, the user will be able to identify the equipment/instrument and to enter its serial number. A checkbox is provided to indicate whether the equipment as active or inactive. Any modification by the user to the information will also be tracked by date and user.

The “Equip/Instru” tab allows the user to define the expected equipment or instrument usage information. Drop-down menus are provided for the user to select the inventory location and storage department. The user may select a specific department or location, or he or she may choose the “all” option for both. The user can enter the expected usage value as well as the quantity of the equipment or instrument. In the purchase price textbox, the user can enter the numeric purchase price for the equipment. Based on these values—the expected usage, quantity and purchase price, appropriate pricing for the equipment based on usage. In this screen, the user can also enter the salvage value of the equipment or instrument.

The user can define the pricing for the equipment or instrument in the “Pricing” tab for the Inventory (Equipment/Instrument) screen. In one embodiment, the user prices the equipment/instruments based on the mark up percentage from cost pricing strategy. The user can enter the mark-up percentage in the textbox provided. Cost for equipment/instrument is defined by the formula PURCHASE PRICE/EXPECTED USAGE. For example, if the percentage is defined as 110%, then the pricing is arrived at under the formula (110/100)*(PURCHASE PRICE/EXPECTED USAGE).

The “Patient Tx” tab allows a user to view patient transactions by reason code for a specific equipment/instrument. The filter provided in this tab allows the user to enter the patient name, reason code and serial number and to search by any of these criteria. The search result is displayed in the bottom frame by the transaction date, patient, reason code and serial number.

### Adding Vendor Information

The next step in setting up is to add vendor information. The user clicks on the Vendors icon to navigate to the Vendors screen. The screen provides a filter to allow the user to search for specific vendors. Textboxes for vendor's name, account number and contact person are provided to assist the user's search. Results are returned in a list in the bottom frame by vendor account number, contact person and telephone number. The user can drill down on an entry by double-clicking on it. The “Vendor Details” screen appears when the “New” radio button is clicked. The Vendor Details screen is divided into the “General Information” tab, the “Products” tab and the “Account” tab. In the General Information tab screen, the user enters the basic vendor information including account number, contact person, address, telephone, fax number, email and website information in the provided textboxes. A checkbox is provided for the user to indicate if the vendor is active or inactive. In the Products tab, the vendors' available products are listed by inventory item name, vendor item number, manufacturer's item number, unit quantity (Qty/UOM), cost/unit, manufacturer, and optionally the procurement contract expiration date. The user may add to this list manually or through the vendors' electronic catalog, as described below. Optionally, a url link to the vendor's electronic catalog is available for the user to click on, which will then direct the user to that web page in a new browser. Where a product is no longer purchased through the vendor, the user may remove the product from the vendor's product list by clicking the delete button on the right-most column. In the Account tab, textboxes are available for the user to input the practice's account number with the vendor, the terms, Tax ID number, credit limit and balance. The user may also click on a check box to show that the vendor is eligible for 1099. Without the necessary information in the Account tab, the user will not be able to order items from the vendor.

Once the information is saved, the vendors saved in the system will appear in a list in the main Vendors screen showing the vendor name, account number, contact person and telephone number. The user may delete vendor contacts by selecting and deleting the entry as necessary.

Based on settings configured under Inventory Preferences, authorized users are able to generate purchase orders with items from the electronic vendor catalog used to populate the products list in the Products tab. In some embodiments of the invention the purchase orders are sent electronically to the vendor; in other embodiments the purchase orders are faxed or mailed to the vendor to place the order.

### Adding Manufacturer Information

In addition to vendors, the user may enter information about the manufacturers of the inventory items. The user clicks on the Manufacturers icon (ADMIN>>Manufacturers) to navigate to the “Manufacturers List” screen. This screen lists all entities that are manufacturers of any of the inventory items in the system by manufacture name, contact person and telephone number. The user may delete a manufacturer contact by clicking the delete icon on the right-most column. Double clicking on an entry or clicking the “New” button directs the user to the “Manufacturers' Details” screen where the user may edit information about the manufacturer. Textboxes are available for entery of the manufacturer's name, address, contact person, telephone and fax numbers, and email and website. Optionally, a search screen can be provided to enable a user to search the manufacturers' records by manufacturer name or by contact person.

If the user's clinical practice has a direct purchase relationship with a manufacturer, the manufacturer must be added as a vendor so that the requisition/purchase order system can be used.

### Inventory Adjustment Types

The Inventory Adjustment Type List screen, accessible from the ADMIN section, enables the user to create adjustment types for inventory. Adjustment types are used in multiple modules of the invention: Purchase Order, Encounter Return, Physical Count and intra-departmental transfer. The user may define whether the net effect of the adjustment is positive or negative. The user may also render the adjustment type either active or inactive. In the Inventory Adjustment Type List screen, all adjustment types are shown including the following are the system-defined adjustment types:
Intra Dept. Transfer (+) Positive—the Inventory transfer happens between two inventory department's when one department adds its inventory to the other department's inventory.
Intra Dept. Transfer (−) Negative—this adjustment automatically occurs to the corresponding Intra Dept. Transfer (+) department above which has a net loss in inventory.
P.O. received (+) Positive—the user receives a P.O. and updates the inventory department.
Physical Count (−) Negative—when a positive physical count variance occurs (Count>On Hand) and the user updates the inventory based on the physical count. This adjustment happens when the physical adjustment count shows more inventory than in the system.
Physical Count (+) Positive—when a negative physical count variance occurs (On Hand>Count) and the user updates the inventory based on the physical count.
Encounter Usage (−) Negative—When the preference card has held onto remaining items which are never used.
Encounter Return (+) Positive—This happens when user actually adds the item to the Preference card.

Accordingly, each inventory adjustment type is designated a net effect that is either positive or negative. The user may designate the adjustment as active by checking the box in the “Active” column. In the right-most column, the delete icon allows the user to soft delete the adjustment type (where it continues to exist in the database for historic purposes) and to remove it from the screen. In another embodiment of the invention, the Adjustment Type List screen further comprises an alpha filter.

Clicking on the “New” button will open the Inventory Adjustment Type Details screen so that the user may add a user-defined adjustment type. Double-clicking on any adjustment in the Inventory Adjustment Types List screen will open a pop-up window for editing the user-defined adjustment type. The user enters an adjustment name in the adjustment name textbox that is unique; i.e., one that is distinguishable from the existing adjustments. The user enters a description of the characteristic of the Adjustment Detail in the text box; and defines whether the net effect is positive or negative. If the net effect is positive, then the inventory count should be added to the inventory. Conversely, if the net effect is negative, the inventory count should be subtracted from the inventory. For the newly-created adjustment to appear in drop-down menus, the user must define it as active.

### Physical Inventory Count

Periodically, the user undertakes a physical inventory count to reconcile the actual on-hand inventory with the information in the system. The following components aid in that process.

The main “Physical Count List” view screen shows a list all previous physical counts and variance reports. The list information includes the count date, the inventory department, storage location, total variance, total monetary variance, the person initiating the count, and the status of the count. An icon is provided as a short cut to the variance report of that physical count process. Through the Physical Count Filter, the user may sort the data by person initiating the physical count, the date ranges of the report; and status of the Physical Count Worksheet.

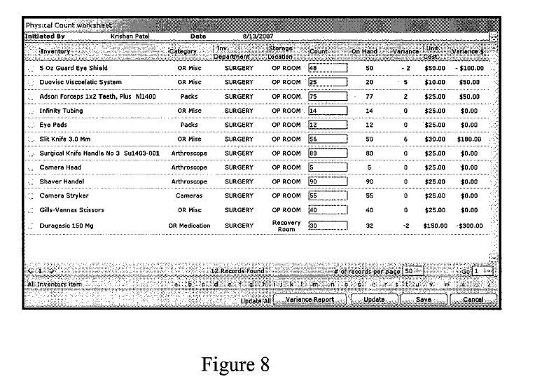

To initiate a new physical count, the user with rights clicks on the “Initiate” button on the upper right corner of the “Physical Count List” view screen. The user is directed to the “Initiate Physical Count” screen. The date and ‘initiated by” fields will be populated automatically and cannot be changed. Drop-down menus for both the inventory department and storage location are populated with all the departments and locations available in the system to facilitate the user's choice. By clicking on the checkboxes for “Include inactive item” and “Include unit cost,” inactive inventory items and the unit cost of the inventory will be included in the physical count worksheet, respectively. The screen also includes an inventory categories multi-select list-box. All the inventory categories in the inventory department and storage location are listed. When the user clicks on the “initiate” button, the user is directed to the Physical Count Worksheet screen (FIG. 8).

The Physical Count Worksheet screen is labeled with the date and name of the individual initiating the physical count. The inventory list shows the names of all inventory items in the storage location for the physical count. The category, inventory department, storage location, on-hand computer count and unit cost information are displayed automatically. The user enters the numeric figure results of the physical count (under “Count”), which will be compared to the on-hand computer count number. The results are displayed in the variance column (Variance=Count−On Hand). The monetary variance (Variance $”) of each line item is automatically calculated by multiplying the variance number with the unit cost (Variance*Unit Cost). The user can update the inventory item count by checking the box to the left of the item name and clicking “Update,” which will update the inventory item's quantity using the physical count number. To adjust the inventory count of all the items listed, the user can check the “Update All” box and click the “Update” button. This will update all inventory items in the Physical Count Worksheet using the numeric figures of the physical count as the inventory quantity. Simultaneously, the system will automatically create individual inventory adjustment records for each inventory item. When the user finishes the Worksheet, the user will select the “Complete” checkbox and click the “Save” button to save the Worksheet results as well as changing its status to “Complete.”

### Creating Purchase Orders

To generate a purchase order manually, an authorized user logs in to the system navigates to the Purchase Order screen (ADMIN>>Purchase Orders). The main screen of the Purchase Order page provides a list of recent purchase orders that have been generated within the past 30 days. Each entry is shown by the order number, the user who created the purchase order, the order date, the vendor, and the order status. The list may be sorted by any of these criteria. In one embodiment of the invention, the icons in the status column of the Purchase Order list indicate the transaction (Tx) status and the tracking information:
[symbol] —indicates that the purchase order was electronically sent and received successfully and that the order is still on “Order” status;
[symbol] —indicates that the purchase order was not successfully sent by electronic means. Optionally, an message box will pop up to detail the error when the user rolls the mouse over the icon;
[symbol] —indicates that the purchase order was electronically sent and received successfully and that the Order was received and paid; and
[symbol] —is the icon for tracking numbers information. The user may click on the icon to activate a pop up window (Fig. Screen 32) in which the user can enter the tracking information for the packages.

The user may also search for a particular past purchase order by inputting some information about the order in the Purchase Order Filter such as an order status, vendor, name of the user who made the order (“ordered by”), order date, order date range, inventory item by either vendor item number or manufacturer's item number. In other embodiments, the Filter includes checked boxes that enable the user to screen out active or inactive items selectively from the inventory. The user can print out any of the P.O.s listed by clicking a print view icon that appears in the row of the entry. Optionally, the user may remove the P.O. entry from the list by clicking the delete icon.

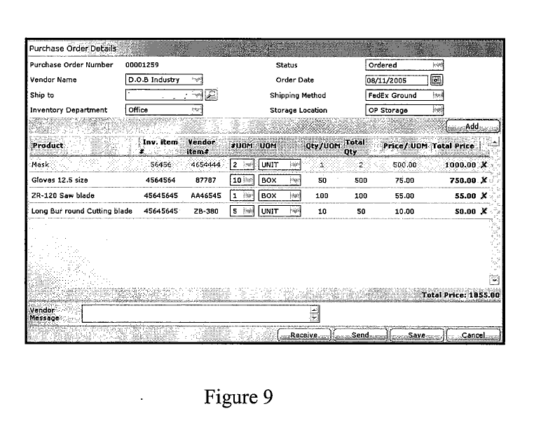

To create a new purchase order, the user begins by clicking the “New” button in the main screen. A new Purchase Order screen titled Purchase Order Details (FIG. 9) will appear, bearing an automatically generated purchase order number and showing by default the preferred inventory department and storage location. The department and storage location information may be changed as necessary by selecting other options in the drop-down menu. The user then selects a vendor from the vendor name drop-down menu. Upon selection, the product list will be populated with the vendor's available items. The user can select the quantity and unit of the items to be ordered by drop down bars. The user can then enter a numeric value in the UOM column and remove items that the item entry in the purchase order. The user has the option of saving the purchase order for completion at another time, in which case the order status will remain as “Open.” While the status remains “Open,” the user may change any field in the purchase order. In the preferred embodiment, the user completes the purchase order by sending it electronically to the vendor by clicking the “Send” button. Optionally, the user may fax or mail the order to the vendor. Once the order has been sent, the purchase order's status is changed to “Ordered.” When the status of the order is changed to “Ordered,” elements of the purchase order, other than the status, may not be changed. For example, the vendor drop-down menu becomes inactive (grayed-out).

If the user has set up purchase order alerts in the system preferences, then selected users will receive an alert for the creation of the purchase order.

In an alternate embodiment, a comments text box is available for the user to enter text so that comments and instructions may be sent to the vendor. In another embodiment the comments are internal messages about the purchase order. In another embodiment, the user creates the purchase order from the CENTCOM screen and not the ADMIN section.

Only designated users are given permission to create purchase orders. Requisitions are created by those users who are not permitted to create purchase orders to request needed items. The requisition is generated and sent to the user who creates purchase orders for approval and ordering. The component that is not pictured—the “Inventory Transaction Codes”—is the mechanism for updating the quantity of inventory items used in the stand-alone module of the invention. In the EHR model, inventory is tied to the patient transaction and will automatically be debited based on the procedure performed.

### Creating Requisitions

In another embodiment of the invention, other users in the same practice will experience a need for an inventory item. Since these users are not authorized to create purchase orders, they create requisitions to prompt authorized users to generate the necessary purchase order.

The user clicks the “Requisition” icon from the ADMIN section and is directed to the Requisition List screen. This screen provides a list of recent requisitions that have been generated within the past 30 days, showing the requisition number, the inventory department and storage location issuing the requisition, the user who created the requisition, the created date and the requisition status. A “notes” icon is provided to show any comments made by a user about the requisition. Comments are shown by rolling the mouse over the notes icon. A Requisition Filter is provided for the user to search for a particular requisition by the created date range, the person who created the requisition (“Created by”), modified by, inventory department, storage location and requisition information in the Requisition List Filter. The search result appears in a list in the bottom frame.

To create a new requisition, the user begins by clicking the “New” button in the main Requisition screen. The “Requisition Details” screen will appear labeled with an automatically assigned Requisition number. The default preferred inventory department and storage location appears on the selection in drop-down menus, but they may be changed as needed. The user enters his or her name from the drop-down box under “Requisition To.” The user can request any amount of the inventory item by entering the numeric value in the quantity (Qty) needed box. Once completed, the user changes the status of the requisition to “Open.” Comments about the P.O. can be added in the Message textbox. In the preferred embodiment, a user who is authorized to process the requisition and create purchase orders receives an alert based on intelliflow rules that a requisition has been created.

In an alternate embodiment, the user creates the requisition from the CENTCOM screen and not the ADMIN section. In yet another embodiment, a requisition is generated automatically based on preferences set up. When an inventory item reaches a pre-defined critical volume or reorder point, a requisition is automatically generated.

### Intelliflow Alerts

The inventory module of the invention provides a system of alerts for the requisition and purchase order process. The alert appears on the CENTCOM task to do list. The user is able to define and modify which individual receives the alert and the task.

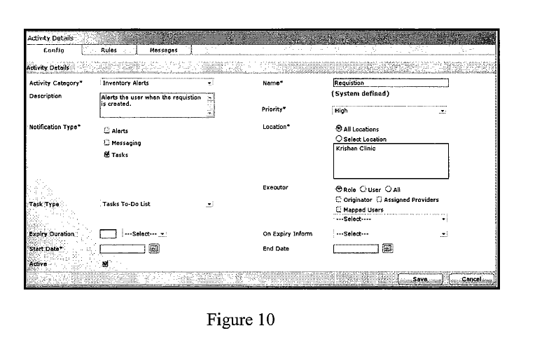

From the ADMIN panel, the user goes to the “Intelliflow” section and navigates to the “Activity Category” screen which lists all the existing Activity Categories including calendar appointments, lab results, and inventory alerts. To set the alerts, the user clicks on “Inventory Alerts” and is directed to the Activities Details screen. The screen is divided into configuration (“Config”), Rules and Messages tabs. Under the Config tab (FIG. 10), the user enters the description of the alert or the task and names the alert. The user also defines the notification type (alerts, messaging, or tasks), the priority, the location (all or a specific location), task type (to do list), and the executor (role and name). The user selects the checkbox Active to activate the alert/task. If applicable, the user can provide a start and end date and/or an expiry duration. The user may optionally select an individual to be notified at the expiration of the alert or task duration.

Under the messages tab, the user can create the message that is sent out when the alert goes out. The message can describe the event succinctly or include more specific instructions. In the preferred embodiment, the message subject and body will either both be “Requisition creation” or “Purchase Order creation.” The alerts will appear in the CENTCOM Task To Do List Summary View Box (“SVB”). A requisition alert will display the requisition as “Requisition—(name of requester)”. A purchase order creation alert will show the name of the vendor instead. The user can roll his or her mouse over the icon to see the requisition or p.o. number. The requisition or p.o. can be accessed by double-clicking on the icon.

The Requisition Task Screen can be accessed by clicking on the text of the requisition alert in the CENTCOM To Do List screen. Rolling over the icon on the top right of the screen will reveal the requisition number, and double clicking the icon will direct the user to the requisition itself. The task screen shows who the task was assigned by, the name of the task and what type of task it is, the priority of the task, when it was created and when it is due. The task description the Requisition shows the message “Requisition creation”, the person creating the requisition, the requisition number, the inventory department and the storage location for which the requisition was made. The user can change the status of the task, the completion date and who should be notified of the task's completion by drop-down menus selection. In addition, the user can enter remarks and the description of the action taken.

The features of the two screens are the same and the following discussion on the Requisition Task Screen applies equally to The Purchase Order Task screen. The task description for a Purchase Order will show the message, person creating the purchase order, the PO number, the vendor to whom the PO has been placed, and the inventory department and the storage location for which the purchase order was created.

### Receiving Inventory

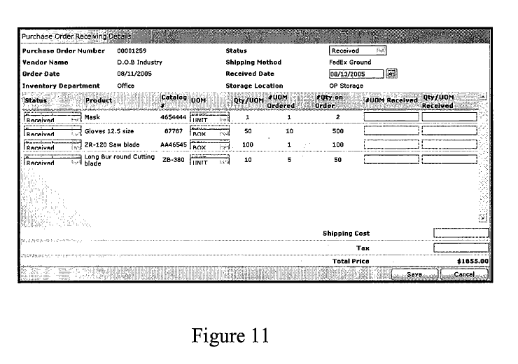

When a shipment is received, the user can rely on the Purchase Order to check in the inventory items. The User navigates to the Purchase Order Screen and locates the applicable Purchase Order through the Purchase Order Filter. The user then clicks the “Receive” button, which directs the user to the “Purchase Order Receiving Details” screen (FIG. 11). Where the status of the purchase order is either RECEIVED, PARTIALLY RECEIVED, or INCOMPLETE, the user may also reach the “Purchase Order Receiving Details” screen by double-clicking on the purchase order record.

In that screen, the Purchase Order number, the vendor name, order date, inventory department, storage location and shipping method will automatically be populated. The user will enter the Received Date for the Order in the textbox in the top frame. For each line item received, the user will enter the quantity and unit received. If the item is serialized, the user will be prompted to enter the serial numbers for the item. The user will change the status of the item as applicable to “Received,” “Partially Received,” “Incomplete” or “Cancelled.” Based on the actual items received, the user will change the status of the Purchase Order in the top frame to “Received,” “Partially Received,” “Incomplete,” or “Cancelled” using the drop-down menu. Once all items have been received, the user can complete the Purchase Order by changing the status to “Complete” or “Paid.”

In another embodiment of the invention, as part of Receiving, the user can enter the shipment method and tracking number for the Purchase Order. In the preferred embodiment, the user can enter up to five tracking numbers for a shipment. Where the user is receiving items that are serialized, the user can click the serial number icon on the PO Receiving screen to enter the items' serial and lot numbers in the Serial Number entry pop-up screen. In an alternate embodiment, the user may receive the shipment for a Purchase Order through the “CENTCOM” screen and not the ADMIN section.

### Status Logic for Requisitions and Purchase Orders

The following explains the status logic for requisitions and purchase orders.

OPEN—This is the status state by default and automatically populates the Status field when the user clicks on the New Purchase Order button or when an automatic purchase order is generated. The user can manipulate the data of the purchase order while order is in this state.

ORDERED—This state occurs when the user explicitly changes the status field in the Purchase Order Details screen or, where a purchase order is automatically generated, when the user hits the send button on the screen. Once the status has been changed to ORDERED, the user cannot change any of the data of the purchase order and can only change the status to CANCELLED.

RECEIVED—When a user clicks the “Receive” button to check in an order and is directed to the Purchase Order Receive screen, the Status field will automatically display the RECEIVED state. This denotes that the user has received that particular purchase order.

PARTIALLY RECEIVED—If during receiving the user discovers that not all items ordered has been delivered, and the vendor has acknowledged that the order was short, the user may change the status to this state in the Purchase Order Receive screen.

INCOMPLETE—If during receiving the user discovers that not all items ordered have been delivered, but the vendor has indicated that the entire order was shipped, the user may change the status to this state in the Purchase Order Receive screen.

CANCELLED—This is status indicates that the order was cancelled. The user may change the status of a purchase order to this state at any time during the life of the purchase order except when the status is OPEN.

PAID—This status is available as a choice only after the order status has been either RECEIVED or INCOMPLETE.

### Creating Preference Cards

To promote efficiency in preparing for patient encounters, preference cards containing information about the physician, the inventory needs for a particular chief complaint or reason code, or both, can be created and used.

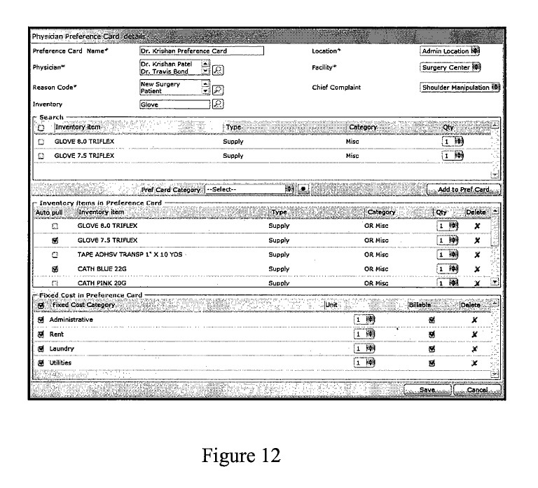

The user navigates to the Preference Card icon and clicks on it to be directed to the “Physician Preference Card” list view. The screen shows all of the preference cards that have been created for the system by card name, location, physician, chief complaint, and reason code. The user can create a new card by clicking the “New” button. The user is transferred to the “Physician Preference Card Details” screen (FIG. 12). The user names the new preference card, or edits the name of an existing one as necessary. From a drop-down menu, the user selects the name of the location and facility for the preference card. The user must associate at least one physician and at least one reason code on the preference card. The user then selects or edits the chief complaint for the preference card. The user can search for inventory items through the search boxes in the top grid of FIG. 12. The user can search by physician, reason code, chief complaint, location, facility, and inventory. The results will be displayed in the “Search” result box. The user then selects and adds at least one inventory item to the preference card by checking the box next to the inventory line item and clicking “Add to Pref Card.” Once added, the inventory items appear in the middle grid titled “Inventory Items in Preference Card.” The lower grid—“Fixed Cost in Preference Card” shows the existing fixed cost as set up in the preference card. The lower grid shows the fixed cost category, the unit, and whether the items are billable (taken from the default from the costing screen). The user's selections are carried forward to the Intellicharge System.

An existing card may be put into edit mode by double-clicking on the entry. To further promote administrative efficiency, an existing preference card may be copied by clicking the “Copy” icon and used as a basis for a new preference card.

To avoid duplication, as the user finishes creating or editing the preference card, he or she will be prompted to further edit the preference card if another card with the exact or substantially similar options exists.

In another embodiment, an autopull check box is provided so that when checked, the preference card items will automatically be added to the charge entry screen.

The user can also add or edit the category for the preference card. From the “Physician Preference Card Details” screen, the user can click on the icon next to the Pref Card Category drop-down menu to create a new preference card category. When the icon is clicked, the “Inventory Preference Card Category Details” screen pops up where the user can enter a new category name and save it as a new category.

### Centcom

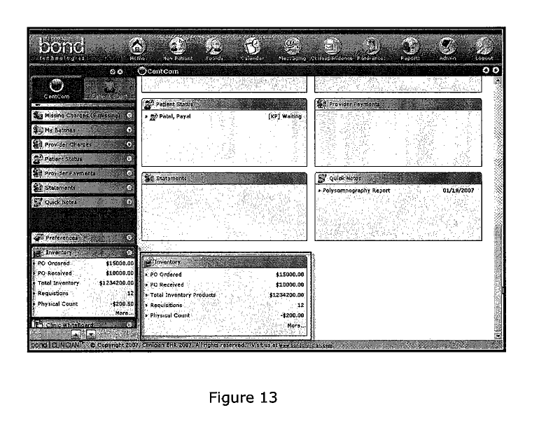

FIG. 13 shows the Central Command screen which is the main user navigation screen. The three boxes in the center frame of the screen are summary view boxes (SVBs). Contained in each SVB are links to the respective entries. A user may drill down upon each link to access the details of the transaction. The Inventory SVB contain links to PO Ordered, which shows all items ordered in the last month; PO Received, which shows all items received in the last month; Total Inventory Products showing what items are sitting on the shelf; Requisitions, which show what items have been ordered by what department; and Accounts Receivable, showing what products have not been paid. The Vendor SVB summarizes how much product was purchased from each vendor distributor in the past month. The user has the ability to view discrete information on individual products and prices. Alternative time periods can be incorporated into the system.

In one embodiment of the invention, the CENTCOM screen already exists as part of the underlying patient management or Electronic Health Record system. The screen needs to be configured to add the inventory module interface of this invention so that users can view a snapshot of the inventory information in the CENTCOM Summary View Boxes (SVBs). The top frame of the screen lists all possible components that have functionality in the CENTCOM screen. The bottom frame contains options for arranging the CENTCOM screen: two windows contain the selected functionality from the top screen; and the user has the option of customizing the order the functional windows would appear in the CENTCOM screen. In the top frame, the user checks the box in the “Show” column next to “Inventory” component so that the inventory module can be added to the functional options for the CENTCOM screen. In the “Arrange CENTCOM” section in the bottom screen, “Inventory” appears as an option in one of the two columns. The user can choose to move the inventory window up or down, and from the left column to the right column.

Similarly, configuration is also needed for the PATIENT CHART screen. In the Patient Chart configuration screen, the top frame again shows all possible components. Inventory is checked as one of the components that will be shown. In bottom frame's “Arrange Patient Chart” window, the selected components are split into three columns. The user can customize the order and position of the components. FIG. 13 shows the CENTCOM screen as viewed by a user. The CENTCOM Summary View Boxes (SVB) for inventory appears in both the left panel and the right frame of the screen. By expanding the SVB link, the user is able to see the same information in the left panel inventory box as in the inventory window in the right frame. The inventory SVB shows the cost for POs ordered, POs received, the total system inventory monetary amount, the number of Open requisitions, and the monetary variance between the physical count and the on-hand computer count.

### Inventory Reports

Another feature of the invention is the ability to generate “Inventory Reports”. Types of reports that can be generated are: Adjustment History of Inventory reports, Order Analysis reports, Supplies Received and Used Analysis reports; Unused Supplies reports, Inventory On-Hand reports, Purchase Order List reports; Inventory Reports, Implant Log reports, Supply Conflict Check reports and Encounter Costing Reports.

The “Adjustment History of Inventory” screen allows the user to configure the Adjustment History report. The screen is divided into three areas: the top banner, the left frame and the right frame. The top banner includes textboxes for a date range (from date and to date). A drop-down menu for “Group” is provided so that user can select whether to group results by inventory department or adjustment code. The left frame is for indication of storage location. The user can limit the report to only show transactions involving any storage location that the user has defined. For example, by choosing the checkboxes the user can limit the report results to any operating rooms, only the back office, or only the pre-operation nurse station. The right frame allows the user to limit the type of inventory adjustments that will be captured in the report. By checking the boxes, the user can limit the report to show only any or any combination of Adjust Quantity (+), Adjust Quantity (−), Manual Error Adj (+), Manual Error Adj (−), P.O. Received.

A Report is generated by clicking “Show Report.” The report date is shown on the top of the report. The date range specified by the user is then shown under the banner “Adjustment History for Inventory.” The report lists entries by inventory name, the Adjustment Code, the User, the Transaction Date, the Change in Quantity, and the Value Change. Where the user has chosen to group results by inventory department, the report will be divided by inventory departments and then sub-divided by storage locations. A subtotal for the inventory department is given at the end of the listing for that department. The total value change is given on the bottom of the report. Optionally, a P.O. number may appear next to the entries for adjustment type P.O. Received.

The configuration screen for “Purchase Order List” report is similar to the Adjustment History configuration screen above. The screen is divided into three areas: the top banner, the left frame and the right frame. The top banner includes textboxes for a date range (PO Order date from and PO Order Date to). A drop-down menu for “PO Status” is provided so that user can capture any combination of OPEN, CLOSED, INCOMPLETE, PARTIALLY COMPLETE, or PAID purchase orders in the report. The user can also click on buttons to choose between a summary report or a detailed report. The left frame is for indication of storage location. The user can limit the report to only show transactions involving any storage location that the user has defined. For example, by choosing the checkboxes the user can limit the report results to any operating rooms, only the back office, or only the pre-operation nurse station. The right frame allows the user to limit the vendor P.O.'s that will be captured in the report. By checking the boxes, the user can limit the report to show only P.O.s with any or any combination of the practice's existing vendors.

A Report is generated by clicking “Show Report.” The report date is shown on the top of the report. The date range specified by the user is then shown under the banner “Purchase Order.” The report lists entries by inventory name, catalog number, status, UOM, Price/UOM, Qty Ordered, Cost Ordered, Qty Received, Cost Received, Qty Pending and Cost Pending. The report results are divided by inventory departments and sub-divided by vendors and P.O.s. The tax value, shipping and handling value and the total amount for the P.O. are also shown.

The “Inventory On Hand” configuration screen is also divided into the left frame and the right frame. The left frame is for indication of storage location. The user can limit the report to only show inventory items in any storage location that the user has defined. For example, by choosing the checkboxes the user can limit the report results to any operating rooms, only the back office, or only the pre-operation nurse station. The right frame allows the user to limit the inventory type that will be captured in the report. By checking the boxes, the user can limit the report to show any combination of inventory types.

A Report is generated by clicking “Show Report.” The report date is shown on the top of the report. The banner “Inventory On Hand” identifies the type of report. The report lists entries by inventory item name, inventory code, on hand count, unit price and value on hand. The report results are divided by inventory departments and sub-divided by inventory type. The total on hand count, unit price and value of the inventory for that inventory department are also shown.

The “Encounter Case Costing” configuration screen is divided into four areas: the top banner, the left frame, the center frame and the right frame. The top banner includes textboxes for a date range (date from and date to). The user can also click on buttons to choose between group report or an individual report. Buttons are also provided for the user to choose between an Encounter report (actual values), a Pref Card report (estimated values), a detailed report, and a totals only report. A text box for entry of a patient name is also provided. The left frame is for indication of location. For example, the user can limit the report to only show transactions involving the Admin location or the Surgery location. The center frame lists insurance carriers and allows the user to limit the transactions to those billed to any or a combination of insurance carriers. These carriers are those that have been added to the Carrier tab of the Insurance Directory screen of the ADMIN section. The right frame allows the user to limit transactions with particular physicians that will be captured in the report.

By clicking “Show Report,” the user is directed to a screen listing encounters that can be included in the Encounter Costing Report. The entries are listed by encounter date, complaint, patient name, provider and amount. The user can select the checkboxes next to any or a combination of these entries to be included in the report. Once selected, the user clicks on “PrintQ” to generate the Encounter Costing Report. The report date is shown on the top of the report. The banner “Encounter Costing Report” identifies the type of report. Underneath the banner, the date of service, encounter type, attending physician, the patient name, patient's date of birth, and insurance carrier are shown. The report lists entries by costing type (inventory type, subdivided into inventory name), the Qty/time, the unit cost, and the extended cost. The subtotal for the different inventory types are given after each inventory type section and the grand total is given at the end of the Report.

The “Inventory Usage” configuration screen is divided into three areas: the top banner, the left frame and the right frame. The top banner includes textboxes for a date range (date from and date to). A drop-down menu for grouping by inventory type and storage location is provided for the user to narrow the results. A textbox is provided for the patient name. The left frame is for indicating the inventory type. By checking the boxes, the user can limit the report to show any combination of inventory types.

An Inventory Usage Report is generated by clicking “Show Report.” The report date is shown on the top of the report. If the user has chosen to group the entries in the Report by inventory type, the report will list entries by inventory item name, inventory type, quantity, the transaction date, the person conducting the transaction (Tx By), and the value of the inventory. The report is divided into sections by inventory department and subdivided by patient. If the user has chosen to group the entries by storage location, the report will list the entries by inventory item name, patient name, quantity, the transaction date, the person conducting the transaction (Tx By), and the value of the inventory. The report is divided into sections by inventory department and subdivided by storage locations and inventory type. The total quantity and total value are displayed after each section. At the end of the report, the total quantity transacted and the total value of the inventory item are also shown.

Another feature of the module of the invention is a variance report. The variance report can be accessed from the Physical Count View screen. The report shows the date of the report on top, with a label “Variance Report.” The report date and the person who initiated the report are listed. The body of the report is divided into Inventory departments and subdivided into storage locations. The entries are listed by inventory item name, category, count (physical), on-hand count, variance, unit cost and variance amount. The total variance and variance amount is shown after each section. The total variance and total value change is displayed at the end of the report.

Another feature of the module of the invention is a requisition report view. The report shows the date of the report on top, with a label “Requisition.” The requisition number, requisition date, the person who made the requisition, the inventory department, and the storage location are listed. The body of the report lists the inventory items in the requisition. The entries are listed by inventory item name and requested quantity. A message section appears at the bottom of the report.

The “Supply Conflict Check” feature enables the user to guard against scheduling appointments when there is a supply conflict and equipment, supplies or device is not available. The configuration screen is divided into three areas: the top banner, the left frame and the right frame. The top banner includes textboxes for a date range (from appointment date and to appointment date). A drop-down menu for grouping by inventory department is provided for the user to narrow the results. The left frame is for indicating the storage location. The user can limit the report to only show inventory involving any storage location that the user has defined. For example, by choosing the checkboxes the user can limit the report results to any operating rooms, only the back office, or only the pre-operation nurse station. The right frame lists inventory types that the user can select to limit the search results. By checking the boxes, the user can limit the report to show any combination of inventory types.

By clicking the Show Report button, a Supply Conflict Check report is generated. The date of the report appears on top, as does the date range entered by the user. The report is divided by inventory department and subdivided into inventory types. The items are listed by inventory name, on-hand count, number needed, short number, on order number (on P.O.) and in other department. The user can therefore see the availability of the equipment, supply or device in another department. In another embodiment of the invention, a time range drop-down menu is provided so that the user can check for conflicts in a given time period. Pre-determined ranges will be provided in the drop-down menu: tomorrow, 1 week in advance, 2 week in advance, 3 week in advance, and 1 month in advance. In yet another embodiment of the invention, the system can provide an analysis of what medical inventory items or pharmaceuticals are being used by which geographical region.

### Making Appointments and Pulling Inventory

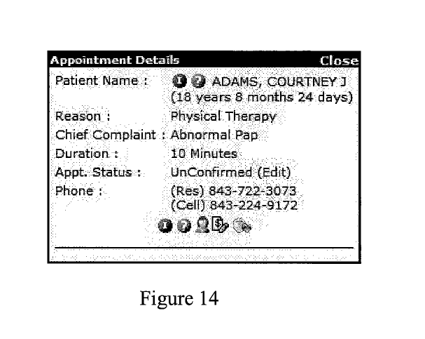

The inventory module interface helps the user ready for patient encounters. The user logs in to the calendar module of the patent management application to book an appointment for a patient. The user follows the steps to make an appointment by entering the required information. Based on the location, facility, physician, reason code and chief complaint information entered, a calendar tool tip helps the user access the appropriate preference card to prepare for the appointment. FIG. 14 illustrates the Calendar Tool Tip with inventory icon. If none of the preference cards match the appointment details, then the icon will not appear. If multiple preference cards match the appointment details, all inventory associated with the preference cards will be aggregated in the inventory list.

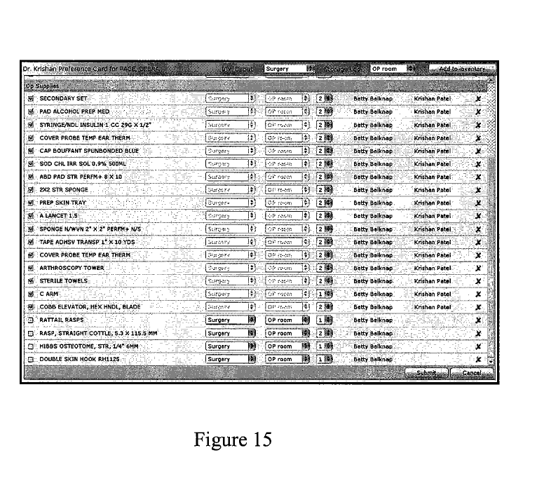

The preference card screen (FIG. 15) will automatically show all the inventory items associated with the default department and location. The inventory department and storage location information is automatically defaulted to the pre-defined preferences. However, the user may add other departments and location, and therefore the inventory items associated, by changing the drop-down menus. The user then checks the selection boxes for the items required for that patient encounter. At the same time, the user may change the quantity of the items to be pulled. By clicking the “Add to Inventory” button, the items are added to the Preference Details screen and the inventory screen in the patient record. These inventory items will be pulled for the patient encounter. If in booking the appointment, the inventory item demand exceeds the supply available, the user will be notified through an error message.

In one embodiment of the invention, the inventory list information also includes the inventory department, storage location from which the inventory is to be pulled, the user name who pulled the inventory, the user name who checked the pulled inventory. In this embodiment, after an appointment is made a user physically goes and selects the items. The user then checks the check boxes and change the quantity pulled if necessary. After that, the user saves the record of this process by clicking the “Save” button. The items which have been selected (checked) will show the user name as the “checked by” user. The quantity, inventory department, storage location and checkbox will then be grayed out.

### Patient Chart Details

To track the dispensing and usage of medical supplies, DMEs, medical devices and pharmaceuticals to patients, the inventory module of the invention allows users to view the inventory allocation for the patient through the patient chart. The inventory allocation can be viewed by encounter. The user is able to filter the results based on inventory items, serial numbers, reason codes, and date range. The user is also able to view the details of the inventory by simply clicking on the row of inventory item or encounter.

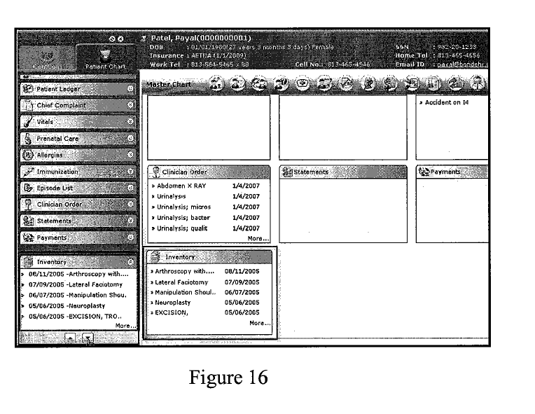

FIGS. 16-18 illustrate the inventory module as it functions in the Patient Chart screens. FIG. 16 shows the inventory SVB in the patent chart and the patient chart links. In the patient chart left frame view the inventory link shows the past five (5) encounters.

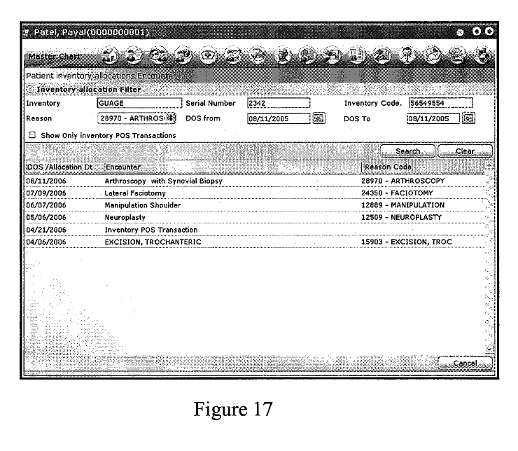

FIG. 17 shows the Patient inventory allocations Encounter screen. The user accesses this screen by clicking on the “Inventory” heading link of the SVB in or the patent chart inventory view heading in the left frame. The Inventory allocation filter shows the filtering options. The user can search by the inventory name, or a partial spelling of the inventory name, in the inventory textbox. Likewise, the user can search for the inventory code, or a partial string of the inventory code, in the inventory code textbox. The drop-down menu for reason codes allows the user to select a reason code and filter inventory allocation records by that parameter. Another parameter available to the user as a search filter is the date range, where the user can enter either the “date from” or “date to” value. The user can further limit the search results to only Inventory POS transactions by checking the “Show Only Inventory POS Transactions” box. Clicking the “Search” button will execute the search. To reset the search parameters, the user will click on the “Clear” button.

The search results will be listed in the bottom frame below the Inventory Allocation Filter. The “DOS/Allocation Dt” column shows the date of service of the encounter. In the Encounter column, the chief complaint of the inventory transactions is displayed. In another embodiment of the invention, the Inventory POS transaction is shown. The reason code for the encounter is also displayed. The user can access the details of any of these transactions by double clicking on the entry.

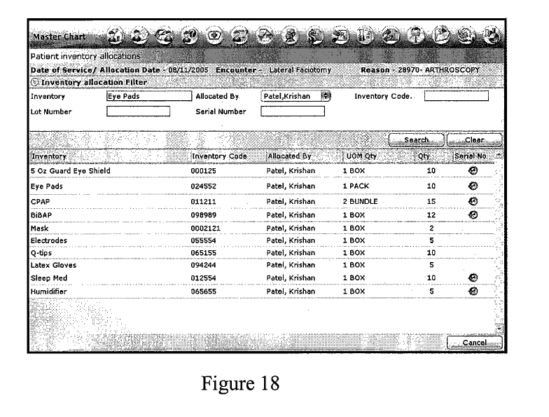

FIG. 18 shows the “Patient Inventory Allocations” screen. The date of service/allocation date, encounter and reason code information will be displayed for the entry. In this screen, another Inventory Filter is provided to enable the user to further narrow the inventory results. The user can search by inventory name or a partial spelling of the name; by inventory code or a partial string of the code. The user can also search by the person allocating the inventory from a drop-down menu selection. The user can search by serial number or lot number of the inventory. The filter results are returned in the bottom frame below the Filter. Each entry is listed by inventory name, inventory code, the “Allocated by” individual, the UOM quantity, the total quantity and the serial number. By rolling over the icon under the serial number column, the serial number will be displayed. In another embodiment, the serial number is displayed directly.

### Point of Sale Inventory

At the conclusion of the patient encounter, the user can use the inventory module interface to assist in checking out. The user logs into the inventory module interface and navigates to the CENTCOM screen. The user then clicks on the Inventory SVB or the inventory link SVB to be directed to the point of sale inventory screen. From there, the user can view the recent point of sale transactions (for the past 30 days). Likewise, he or she can access and view the receipts for recent finished transactions.

In that screen, the user can create a new point of sale transaction. The user selects the inventory, the diagnosis and procedure codes for the inventory transaction. The user may select from either the “Bill to Insurance” or the “Bill to Patient” options. If the “Bill to Insurance” option is selected, a “Charge Capture” screen pops up and will prompt the user to select the CPT code and Dx code per line item. If the user selects the “Bill to Patient” option, then “Retail Payment” screen pops up. In either case, the user will check off the inventory items that were used during the encounter thereby adding the price of the inventory items to the charge. The user then checks the “Charge Complete” box and saves the charge record. The record is now locked and cannot be edited. All the unchecked (unused) inventory items are adjusted for inventory. Once the user finishes the transaction, a summary of it will be displayed on the Summary screen. The user will log out of the patient's record.

In another embodiment, by clicking a link in the left panel, a Point of Sale (POS) Inventory screen appears in the right frame. An Inventory Filter in this screen provides a user with the ability to search for inventory that has been involved in a POS transaction by inventory name, patient, transaction date or date range, and/or inventory code. Results are returned to the bottom frame and are listed by inventory name, patient, transaction date, UOM Qty, quantity (QTY) and type. In this screen, the user is able to tell which inventory items are billed to the patient and which are billed to insurance. Under the column labeled “Type,” items that are billed to the patient are labeled P, and items that are billed to insurance are labeled I. The Inventory Filter enables the user to search for past Point of Sale Transactions. The user is also able to view the receipt of paid items by clicking on the receipt icon next to the “Type” column.

The user clicks “New” and is directed to the Inventory Search/Add screen, where the user can execute a new POS inventory item event. In a search box in the top frame, the user searches for the inventory item by patient (the one for whom the transaction is being done), inventory department, storage location, inventory category and inventory. The user should only select a single inventory department and a single storage location. The search results are displayed in a list, shown by inventory, inventory category, and vendor in the middle frame of the screen. Optionally the storage location may also be shown. The user selects the appropriate inventory items that have been used during the patient encounter by checking the boxes. By clicking the “Add to List” button, the selected inventory items are added to the allocated inventory frame in the bottom. The allocated inventory appears in a list at the bottom of the screen. Inventory is displayed by inventory name, UOM, Qty/UOM, Qty, Total Qty, Vendor, Procedure Code, Dx Code, Cost/UOM and Billed Amount. Each item has its own check box so the user can select payment for a specific inventory item. The user enters the inventory quantity (Qty). The default procedure code and diagnostic code are shown, however, the user may change either or both as appropriate. A Diagnosis Search box is provided. The user can also delete items from this list by clicking the delete icon on the right. Radio buttons are provided for the user to choose whether to “Bill To Patient” or to “Bill To Insurance.” If the option for billing to insurance is chosen, then the user is prompted to select the physician from the Billing Provider drop-down menu.

The “Inventory Payment Screen” is where the patient is billed in a retail transaction. In the top window frame, the items being charged are listed by inventory name, vendor. The items are displayed by columns showing the UOM, Qty/UOM, UOM Qty, Total Qty, Cost/UOM, and Price/UOM. A tax column displays “Yes” when the item is taxable and “No” when it is not. The last column shows the extended price of the item. The user can click on the [symbol] discount icon to access the available discount, if any. A delete icon is provided for the user to delete individual inventory item rows.

The lower window frame of the Inventory Payment screen is divided into three areas. On the left side is the Product Payment window. There the user chooses the payment type: drop-down menus are provided for payment type (for e.g. cash, check, credit card, or other) and textboxes are provided for amounts and other identifying characteristic (for e.g. brand of credit card, check number). At the bottom of the Payment Tender Window a textbox is provided for the user to enter a tendered amount. Below the Payment Tender Window is an memo area with a textbox for the user to enter comments. To the right is the Payment Received window, which has textboxes for the subtotal of the transaction, the order discount, the discounted amount, the tax, the total amount, the amount tendered, and the balance. To create a payment receipt, the user selects a PrintQ checkbox next to the “Pay” button and clicks on “Pay” to process the transaction. Clicking the pay button completes the transaction and closes the window. The transaction will then be included in the Payment Day sheet report and the Encounter Face Sheet report. When the transaction closes, the transaction record is added to the Inventory CENTCOM screen as well as the Patient Transaction tab in the Inventory Details screen. If the user clicks the “Cancel” button, the window closes without completing the transaction.

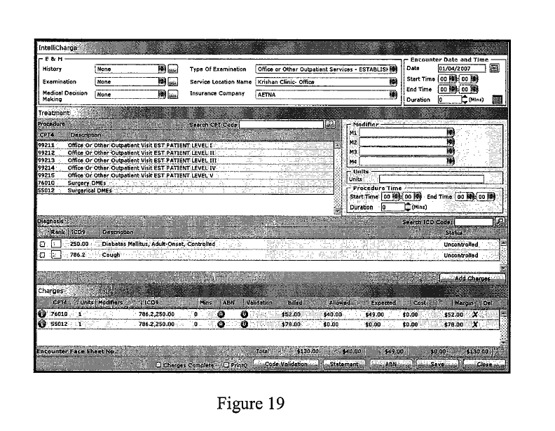

FIG. 19 shows the IntelliCharge screen where the charges are billed to insurance. If the user clicks the “Payment” button after selecting “Bill to Insurance,” the user will be directed to the existing PM/EHR system's IntelliCharge screen. It is a charge capture screen where preferably the CPT codes and diagnosis is automatically copied from the transaction table. The user may also enter the CPT codes manually. Completing the transaction will create a row in the Inventory CENTCOM Screen with a $ icon which, upon clicking will open the Encounter Face Sheet. The transaction will also be included in the Encounter Face Sheet Reports and the Payment Day Sheet report.

### Costing Screen

The cost analysis and capture feature of the invention allows the user to create costing details based on the practice's location. The feature further enables the user to define total unit cost for each fixed cost category using the allocation % formula Unit cost=(Annual cost*(Allocation %/100))/AVG Case per Year. It also allows the user to define the direct labor cost based on the roles users have defined under the user management.

The user navigates to the “Costing List View” screen from the ADMIN section. The screen shows existing costing details for the practice locations organized by location name. The last modification dates and the users who made those modifications are also shown. Delete buttons in the right-most column enable the user to delete the costing entry. By clicking “New,” the user is directed to the Costing Details screen.

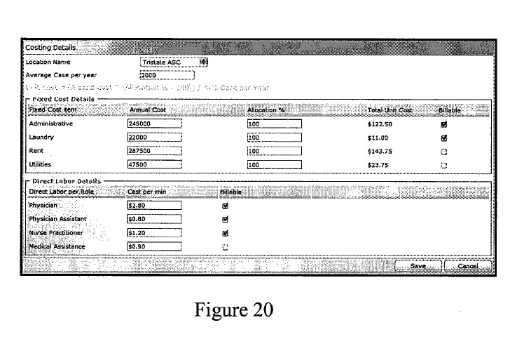

In the Costing Details screen (FIG. 20), the user can define the costing data for a practice location. First, the user selects the practice location name from a drop-down menu that contains all of the location names. The user then enters the numerical value of the average cases per year for the location. In a Fixed Cost Details field box, all items of fixed costs as defined in the Fixed Cost List View screen are shown. The user enters the number for the annual cost for each item of fixed cost as well as the percentage of allocation. The total unit cost is automatically calculated. The user defines whether an item of fixed cost is billable: if the box is checked for the item, the value will be added to the patient bill; if not, the value will only be a factor in the encounter costing report and will not be billed to the patient.

In a Direct Labor Details field box, the roles of direct labor such as physician, nurse practitioner and physician's assistant, as defined in the user management module, are listed in the column “Direct Labor per Role.” The user enters the cost per minute for each of these roles. Each of these costs is also determined to be billable (box checked) or non-billable (box unchecked). Again, only billable costs will be added to the patient bill.

### Fixed Cost Categories

To capture the fixed cost, the user first defines the fixed cost categories. In the “Fixed Cost Categories” screen, the user is able to view the existing categories of fixed costs. The user is able to edit or delete existing ones by double-clicking on the category which will activate a pop up window with the category in edit mode. The user can also click on the “New” button to be directed to the “Fixed Cost Details” window to create new fixed cost categories.

### Map Clinician Monitor Status and Location

Another cost capture feature of the invention configures the relation between clinician monitor status and location. The “Map Clinician Monitor Status and Location” screen enables the user to associate cost per clinician monitor status. The user can choose any of the practice locations from the drop-down menu. Mapping status is defined as the waiting room, the pre-op room, the op room, recovery stage-1 and recovery stage-2 rooms. The user enters the numeric value of cost per minute in the “Unit $ cost per minute” text box. By checking the billable box, the cost is included in the patient's bill. If the box is unchecked, the cost is not billed to the patient; it only appears in the encounter costing report.

All displayed lists of the invention may be sorted alphabetically by characteristic by clicking on the column heading. For instance, by clicking on inventory type, the products in the list will be sorted by inventory type alphabetically. A user can likewise quickly access the record of the item of interest by navigating to the screen showing similarly spelled items by clicking the appropriate alphabet letter in the bottom of the screen, or by entering the number corresponding to the record of interest.

While the invention has been described by illustrative embodiments, additional advantages and modifications will occur to those skilled in the art. Therefore, the invention in its broader aspects is not limited to specific details shown and described herein. Modifications may be made without departing from the spirit and scope of the invention. Particular embodiments of the invention may also be applied to other industries. Accordingly, it is intended that the invention not be limited to the specific illustrative embodiments, but be interpreted within the full spirit and scope of the embodiments described herein and their equivalents.

1. A system for inventory management and billing to be associated with a practice management system or an electronic health record system comprising:
an input device for entering data operably linked to a processor;
a memory and a computer-executable program stored in said memory;
at least one server for storing inventory data, scheduling data and multiple vendor catalog data;
a processor for receiving, processing and, transforming incoming data;
a processor for querying the scheduling data, inventory data, the vendor catalog data or patient data stored in a practice management system or electronic health record system;
an output device for displaying the scheduling data, inventory data, the vendor catalog data or patient data; and
at least a computer is connected to a communications network; and wherein the computer, upon executing the program, tracks inventory levels as patient encounters are scheduled and conducted through a calendar module; wherein the computer accesses and modifies inventory data through an inventory module and bills the patient according to inventory used during the patient encounter through a point of sale transaction generated by the inventory module; wherein the computer generates and processes inventory and cost reports; wherein the computer generates and processes a requisition or purchase order at predetermined levels; wherein the computer alerts authorized users upon said requisition or purchase order being generated.

2. The system of claim 1, wherein the computer system is configured to execute a program comprising the steps of:
generating an electronic requisition using an inventory module and accessing inventory information;
querying the server using a processor for vendor catalog data;
processing the requisition by executing the program using a processor;
generating, by executing the program using a processor, a purchase order electronically using information provided by the requisition;
submitting the purchase order to a vendor via electronic means;
receiving a tracking number from the vendor via electronic means;
inputting the tracking number into the inventory module using an input device;
receiving shipment containing items in the purchase order;
receiving a serial or lot number for each serialized item in the purchase order via electronic means;
inputting the serial or lot number for each serialized item in the purchase order into the inventory module using an input device;
checking-in the items using a purchase order receiving component of the inventory module;
updating the inventory information; and
displaying the record on the output device.

3. The system of claim 2, wherein the computer is further configured to track internal departmental transfers of inventory.

4. The system of claim 2, wherein the computer is further configured to execute a point of sale transaction using the inventory module.

5. The system of claim 2, wherein the computer is configured to store information about a plurality of medical inventory items, comprising item prices, on-hand count, unit of measurement, unit price, and cost; and wherein the computer is configured to generate inventory and cost reports.

6. The system of claim 2, wherein the computer is further configured to execute a program comprising the steps of:
setting up at least one electronic physician preference card to prepare for patient encounters, said preference cards containing information about at least one inventory department, at least one storage location, at least one physician, at least one chief complaint, at least one inventory item and at least one reason code;
entering a patient encounter appointment for a patient in the calendar module using the input device;
accessing all suitable preference cards according to the physician, the chief complaint, and the reason code via a processor configured to query an inventory module;
checking for inventory supply conflict;
allocating at least one inventory item by selecting it electronically for the patient encounter;
correcting the allocation by returning unused inventory items after conducting the patient encounter;
saving the allocation record in the patient's electronic chart;
conducting a point of sale transaction using the inventory module by selecting the allocation, the diagnosis and procedure codes;
billing the patient or an insurance carrier.

7. The computer system of claim 2, wherein the computer is further configured to execute a program comprising the steps of:
setting up inventory preferences in the inventory module using the input device;
entering a preferred shipping method in the inventory module using the input device;
entering a preferred costing method in the inventory module using the input device;
entering at least one inventory department in the inventory module using the input device;
entering at least one storage location in the inventory module using the input device;
entering at least one inventory category in the inventory module using the input device;
setting up requisition preferences using in the inventory module using the input device;
entering at least one reorder point for at least one inventory item in the inventory module using the input device; and
selecting an individual to receive alerts of automatically-generated requisitions.

8. A computer system of claim 2, wherein the computer is further configured to execute a program comprising the steps of:
setting up alerts in the inventory module;
entering a task name for one alert in the inventory module using the input device;
entering a task description for the alert in the inventory module using the input device;
entering a notification type in the inventory module using the input device;
entering a priority in the inventory module using the input device;
entering a storage location in the inventory module using the input device;
entering a task type using an in the inventory module using the input device;
entering and designating an authorized user as an executor in the inventory module using the input device; and
configuring a task to do screen; and
displaying the task to do screen via an output device.

9. The system of claim 6, wherein the computer is further configured to execute a program comprising the steps of:
calculating fixed costs in the inventory module by executing a program using the processor;
calculating mapping costs in the inventory module by executing a program using the processor; and
adding said fixed costs and mapping costs to the patient charge at a point of sale transaction using the inventory module.

10. The system of claim 2, wherein the computer is further configured to execute a program comprising the steps of:
entering a physical count number after undertaking a physical inventory count of inventory items to arrive at physical count numbers;
comparing, by executing a program of instructions executable by a processor, the physical count numbers with an on-hand inventory count in the inventory module;
recording any variance between the physical count numbers and the on-hand inventory count; and
making at least one inventory adjustment to correct the inventory on-hand count to the physical count number; and
displaying the inventory adjustment information using the output device.

11. A computer program product, executed by a computer processor, for inventory management and billing to be associated with a practice management system or an electronic health record system comprising:
a computer readable medium having computer readable program code embodied therein, the computer readable program code configured to access and modify inventory data in an inventory module;
computer readable medium having computer readable program code embodied therein, the computer readable program code configured to access and modify scheduling data in a calendar module;
computer readable program code configured to access and to modify patient data in a practice management system or an electronic health record system;
computer readable program code configured to generate point of sale billing transactions associated with clinical services and supplies provided to patients;
computer readable program code configured to access multiple vendor data from a server; and
computer readable program code configured to automatically generate requisitions or purchase orders at pre-determined reorder point of inventory data.

12. A method of inventory management implemented by a program storage device readable by machine, tangibly embodying a program of instructions executable by the machine to perform steps comprising:
generating an electronic requisition by executing an inventory module and accessing inventory information using the machine;
processing the requisition by executing the program using a processor;
generating, by executing the program using a processor, a purchase order electronically using information provided by the requisition;
submitting the purchase order to a vendor via electronic means;
receiving a tracking number from the vendor via electronic means;
inputting the tracking number into the inventory module using an input device operably linked to the processor;
receiving shipment containing items in the purchase order;
receiving a serial or lot number for each serialized item in the purchase order via electronic means;
inputting the serial or lot number for each serialized item in the purchase order into the inventory module using an input device;
checking-in the items using a purchase order receiving component of the inventory module; and
updating the items' inventory information.

13. The method of claim 12, further comprising the steps of:
entering a patient encounter appointment for a patient in the calendar module using the input device;
accessing Physician Preference Cards via a processor configured to query the inventory module;
checking for inventory supply conflict;
selecting items in the inventory module to prepare for the patient encounter using the Physician Preference Cards;
saving an inventory allocation record of the selected items in a patient chart for the patient;
after gathering the items for the patient encounter and conducting the patient encounter, de-selecting all unused items in the record in the patient chart at the close of the patient encounter; and
updating the items' inventory information.

14. The method of claim 12, further comprising the step of: associating the tracking number with the items.

15. The method of claim 12, wherein the generating and processing the electronic requisition is accomplished by the steps of: monitoring an inventory count of at least one item; generating an electronic requisition automatically when the inventory count reaches a pre-defined reorder point; generating an alert to a pre-determined user, adding the requisition to the pre-determined user's task to do list; and processing the requisition.

16. The method of claim 15, further comprising generating inventory reports and cost reports.

17. The method of claim 16, wherein the inventory report is an inventory adjustment report, a purchase order list report, an inventory on-hand report, an encounter case costing report, an inventory usage report, a variance report, a requisition report or a supply conflict check report.

18. The method of claim 12, further comprising the step of alerting authorized users that a purchase order has been generated.

19. The method of claim 12, wherein the electronic means comprises e-mail, batch processing by an electronic data interchange (EDI) server, or facsimile.

20. The method of claim 13, wherein the inventory module is implemented in a practice management application or an electronic health record system.

21. A computer-based method for managing inventory and billing, comprising the steps of:
setting up at least one electronic Physician Preference Card to prepare for patient encounters, said preference cards containing information about at least one inventory department, at least one storage location, at least one physician, at least one chief complaint, at least one inventory item and at least one reason code;
entering a patient encounter appointment for a patient in the calendar module using an input device operably linked to the machine;
accessing all suitable preference cards according to the physician, the chief complaint, and the reason code via a processor configured to query an inventory module;
checking for inventory supply conflict;
allocating at least one inventory item by selecting it electronically for the patient encounter;
after conducting the patient encounter, correcting the allocation by returning unused inventory items;
saving the allocation record in the patient's electronic chart;
conducting a point of sale transaction using the inventory module by selecting the allocation, the diagnosis and procedure codes;
billing the patient or an insurance carrier.

22. The method of claim 21, further comprising the steps of:
providing a processor to query inventory data and cost data in the inventory module;
calculating fixed costs by executing a program using the processor;
calculating mapping costs by executing a program using the processor; and
adding said fixed costs and mapping costs to the patient charge at a point of sale transaction using the inventory module.

23. The method of claim 12, further comprising the steps of:
entering a physical count number after undertaking a physical inventory count of inventory items using an input device;
comparing, by executing a program of instructions executable by a processor, the physical count numbers with an on-hand inventory count in the inventory module;
recording any variance between the physical count numbers and that on-hand inventory count; and
making at least one inventory adjustment to correct the inventory on-hand count to the physical count number.

24. The method of claim 12, wherein the purchase order is automatically generated based on the requisition.

25. A method of inventory management, comprising the steps of:
storing multiple vendors' electronic catalog information in a central EDI server;
providing a processor to query the EDI server for electronic catalog information to identify items of interest;
querying the EDI server for alternate inventory if the items of interest are unavailable;
generating a purchase order by electronic means to order the items of interest;
saving a record of the purchase order into an inventory module;
submitting the purchase order to a vendor via electronic means;
receiving a tracking number from the vendor via electronic means;
inputting the tracking number into the inventory module;
receiving a serial or lot number for each item of interest via electronic means;
inputting the serial or lot number for each item of interest into the inventory module;
receiving shipment containing items in the purchase order;
checking-in the items using a purchase order receiving component of the inventory module; and
updating the items' inventory information in the inventory module.

26. The method of claim 24, wherein the purchase order is entered manually into a computer system or automatically generated by the computer system.

27. The method of claim 12, further comprising the steps of tracking internal departmental transfers using an Adjustments tab interface in the inventory module.

28. A method for responding to a medical product recall comprising the steps of:
receiving notice of the recall;
searching inventory by serial or lot number;
confirming that the recalled product is in the inventory;
searching inventory allocation records for the products;
locating patients to whom the recalled product has been dispensed; and
notifying the patients of the recall.
